(19) United States
(12) Patent Application Publication
(10) Pub. No.: US 2026/0034340 A1
(43) Pub. Date: Feb. 5, 2026

(54) FOOT POWDER APPLICATION DEVICE

(71) Applicant: Austin Rosen, Boca Raton, FL (US)

(72) Inventor: Austin Rosen, Boca Raton, FL (US)

(21) Appl. No.: 18/791,048

(22) Filed: Jul. 31, 2024

(51) Int. Cl. A61M 35/00

(52) U.S. Cl. CPC A61M 35/006

(57) ABSTRACT

A foot powder application device is disclosed, including a housing defined by a top surface, a bottom, and a plurality of sidewalls. An interior of the housing retains the foot powder therein. A lid is provided on the top surface to be opened and closed via a fastener, wherein opening the lid exposes an opening dimensioned to receive the user's foot. A foam base is positioned on the bottom of the interior of the housing.

### TECHNICAL FIELD

The embodiments disclosed herein generally relate to devices for applying foot powder.

### BACKGROUND

It is well known that maintaining one's hygiene is an important part of a daily routine to reduce the likelihood of infection, prevent unwanted bodily odors, and as a means of maintain or improving one's overall health. In particular, maintaining the health and cleanliness of the feet ensures that the feet do not become injured due to infection as well as preventing unwanted foot odors caused by sweat and the growth of bacteria. The feet are prone to developing odors caused by sweat, which provides a beneficial environment for bacteria to grow, especially when the individual is wearing shoes, socks, cleats, work boots, slippers, and other footwear and footwear accessories.

Various products exist which prevent and/or remediate foot odor. In particular foot powders and creams have been developed which prevent the retention of moisture near the feet, control odor, and soothe skin irritation. Many foot powder products include menthol, talc, and zinc oxide as well as other active and inactive ingredients. To apply the powder, the user will pour the powder directly onto the feet or into the sock or shoe where the foot is placed during use. While effective, this application method can result in pouring the powder onto unwanted area and can cause a mess and result in excess and unnecessary waste

### SUMMARY OF THE INVENTION

This summary is provided to introduce a variety of concepts in a simplified form that is further disclosed in the detailed description of the embodiments. This summary is not intended for determining the scope of the claimed subject matter.

A foot powder application device is disclosed, including a housing defined by a top surface, a bottom, and a plurality of sidewalls. An interior of the housing retains the foot powder therein. A lid is provided on the top surface to be opened and closed via a fastener, wherein opening the lid exposes an opening dimensioned to receive the user's foot and or feet. A foam base is positioned on the bottom of the interior of the housing.

The device provides a sanitary means of applying foot powder to the foot by preventing the spillage of the foot powder onto unwanted areas or throughout the environment. The device can be selectively opened and closed to allow easy application of the foot powder by first pouring powder through the insert, followed by the user inserting the foot and tapping to create a cloud of powder enclosed in the environment, resulting in powder settling on and around the foot as well as to enable the user to carry the device without spilling the foot powder.

In one aspect, the foam base includes antimicrobial properties to prevent the growth of microbes within the interior of the housing.

In one aspect, the closed configuration is defined by the fastener being engaged to close the opening, while the open configuration is defined as the fastener being disengaged to enable the user to open the lid. The closed configuration retains the foot powder within the interior, such that the user may carry and transport the device.

In one aspect, opening the lid exposes an elastic cover, and wherein the opening is provided on the elastic cover to fittingly contact an ankle region of the foot.

In one aspect, the elastic cover includes an elastic component defining an edge of the opening.

In one aspect, the elastic component is biased to fittingly engage with the ankle region.

In one aspect, the device includes a carrying handle attached to the housing. The carrying handle may enable efficient carrying of the device as well as enable the user to attach the device to another object.

In one aspect, the housing is constructed to prevent the access of contaminants into the interior of the housing. This may be accomplished by constructing the housing of a waterproof material as well as constructing the housing to be durable.

In one aspect, the housing includes a storage compartment suitable for storing a water bottle or similar container.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a perspective view of the foot powder application device in an open configuration, according to some embodiments;

FIG. 2 illustrates a perspective view of the foot powder application device in a closed configuration, according to some embodiments;

FIG. 3 illustrates a perspective view of the foot powder application device and the carrying strap, according to some embodiments;

FIG. 4 illustrates a perspective view of the foot powder application device, wherein the user has positioned their foot within the interior of the device to apply the foot powder contained therein, according to some embodiments; and

FIG. 5 illustrates a perspective view of the foot powder application device and foam base, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to a foot powder application device (which may be referred to herein simply as “device” or “the device”). The foot powder application device is used to enable a user to apply a foot powder while retaining the powder within the housing of the device, thus promoting a clean and efficient application of the powder. The foot powder composition is retained within the housing of the device, such that the foot powder is not spilled onto unwanted surface or does not become airborne and float onto unwanted surface. This alleviates the common inconvenience of cleaning the surrounding areas when applying the foot powder onto the feet and avoiding waste.

The embodiments include a housing which contains a foam pad on which the user stands when applying the foot powder. The housing contains the foot powder within the interior of the device such that the foot powder is unable to exit the interior of the housing. The housing includes a selectively openable and closable lid. Closing the lid allows the user to transport the device in between uses, while retaining the foot powder within the closed housing. During use, the user will open the cover and position their foot through an opening and into the interior of the housing to apply the foot powder. Maybe insert the poof, cause by a tapping action of the foot onto the pad inside the environment resulting in a comprehensive (nearly) total foot powder coverage?

In some embodiments, the opening is dimensioned to enable the user to insert their foot through the opening, while the perimeter of the opening is similar to the perimeter of one's ankle region. The ankle region may include the ankle, bottom of the leg, and/or top of the foot.

In some embodiments, the housing may be constructed of a waterproof, weatherproof, wind-resistant, and/or durable material to protect the foot powder retained within its interior form the elements. This ensures that the foot powder does not become wet and prevents unwanted contamination which may be caused by dirt, dust, and other materials.

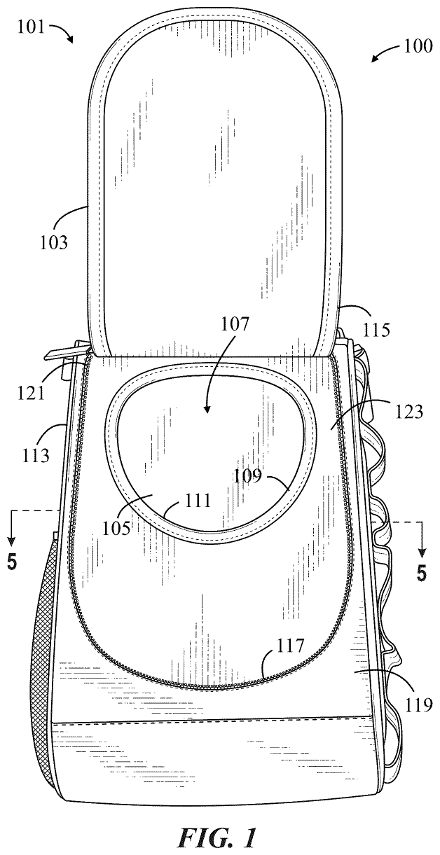

FIG. 1 illustrates a perspective view of the foot powder application device 100 in an open configuration 101. The open configuration 101 is defined by opening the lid 103 which exposes an opening 105 into the interior 107 of the device 100. The opening 105 has an edge 109 defined by a perimeter 111 of the opening 105. The edge 109 may contact the skin of the user or may be near the skin of the user's ankle region such that the foot powder is retained within the interior 107 of the device 100 once the user's foot is placed within the interior 107. The device includes a housing 113 which forms a protective case 115 to retain the foot powder therein and to protect the interior 107, foot powder, and foam pad from the elements and contamination (e.g., moisture, dirt, debris, etc.). A fastener 117 (e.g., a zip fastener, clasp locker, or similar means of connecting two materials) is provided to releasable connect at least a portion of the lid 103 to the top surface 119 of the housing 113. For example, the fastener 117 may be a zipper 121 which has a first zipper component on the lid 103 and a second zipper component on the top surface 119 of the housing 113.

During use, the user opens the lid 103 using the zipper 121 to reveal an elastic cover 123 which is stretchable around the user's ankle region to provide a secure fit around the user's ankle region. The elastic cover 123 includes an elastic component 125 forms the edge 109 of the opening 107. The elastic cover 123 may be bonded or otherwise attached to the top surface 119.

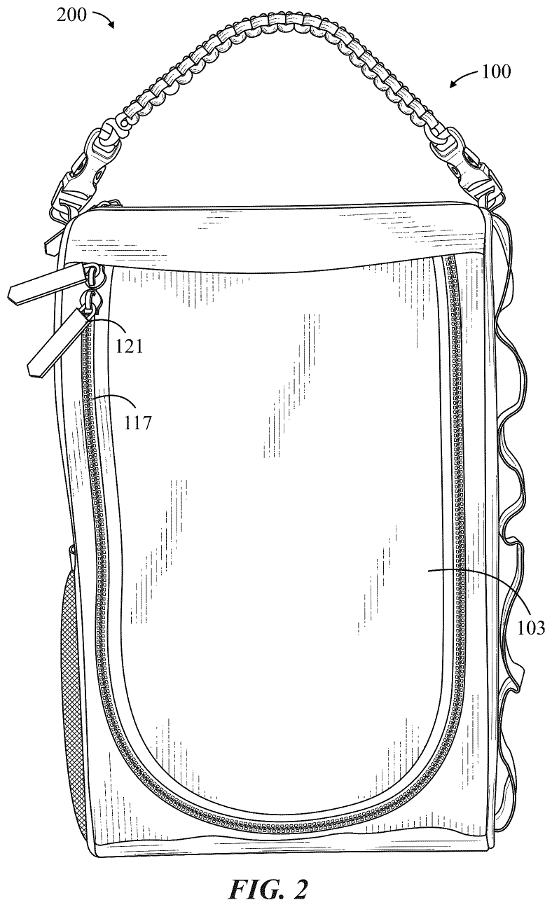

FIG. 2 illustrates a perspective view of the foot powder application device 100 in a closed configuration 200. The closed configuration 200 is defined as the zipper 121 being engaged such that the opening is covered and closed. The closed configuration 200 allows for the user to carry the device 100 and retain the foot powder therein such that it does not exit the interior of the device 100. When the lid 103 is closed (i.e., the closed configuration 200), the elastic cover is covered by the lid 103 thus preventing the foot powder from escaping from the interior of the device 100.

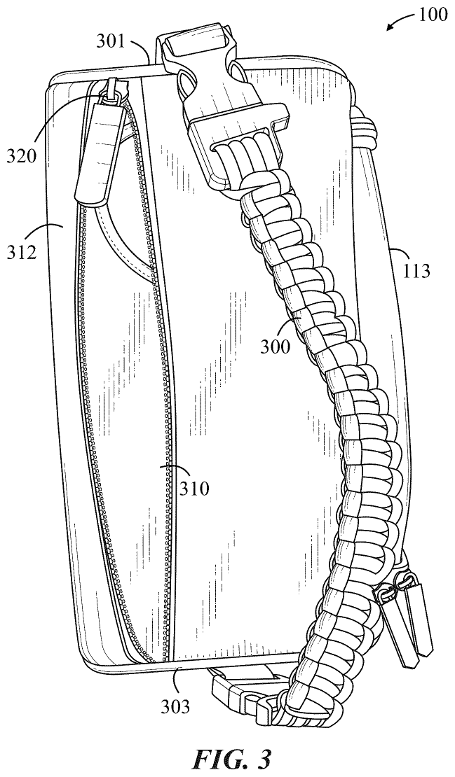

FIG. 3 illustrates a perspective view of the foot powder application device 100 and the carrying strap 300. The carrying strap 300 is connected to the first end 301 and second end 303 of the housing 113 and provides an efficient means for the user to carry they device 100 or interlock the device with another object (e.g., a backpack, purse, bag, etc.) such that it can be carried over periods of time between uses. The device 100 may include a storage pocket 310 to enable the user to store and carry additional items, or additional foot powder therein. The storage pocket 310 may be selectively opened and closed via a second fastener 320 (e.g., a second zipper). The storage pocket 310 is provided on the back 112 of the housing 113.

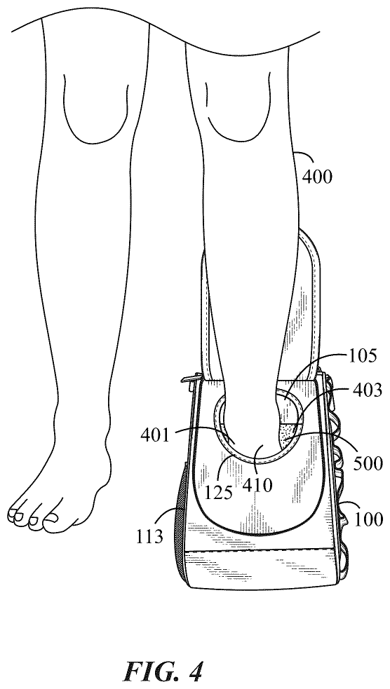

FIG. 4 illustrates a perspective view of the foot powder application device 100, wherein the user 400 has positioned their foot 401 within the interior of the device 100 to apply the foot powder 403 contained therein. A foam base 500 is provided on the bottom of the interior of the housing 113 whereon the user 400 may stand. During use, the user 400 inserts their foot 401 and taps, shakes, or otherwise moves their foot to agitate the foot powder 403 within the device. This provides a sanitary method of applying the foot powder 403 to their foot 401 while preventing the foot powder 403 from exiting the interior of the housing and causing a mess. This process can then be repeated with the other foot if necessary. The opening 105 is dimensioned to fittingly contact the ankle region 410 of the user 400. This is accomplished via the elastic component 125 which is biased to fittingly contact the angle region 410.

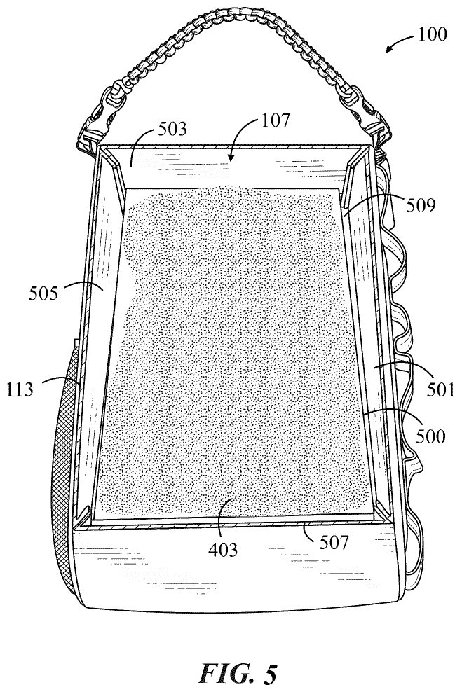

FIG. 5 illustrates a perspective view of the foot powder application device 100 and foam base 500 wherein the top surface and lid have been removed for illustrative purposes to clearly show the interior 107 of the device 100. The foam base 500 is constructed of a foam or other cushioned material which provides a cushioned base 501 for the user to stand upon when their foot is positioned within the interior 107 of the housing 113. The interior is defined by the sidewalls 501,503,505,507 and bottom 509. The foam base 500 is dimensioned to fit between the sidewalls 501,503,505,507 and is placed upon the bottom 509 of the device 100. The foam base 500 may be removable from the interior 107 of the device 100 to enable the user to clean the foam base 500.

In some embodiments, the foam base 500 includes antimicrobial properties to prevent the growth of bacteria on the foam base 500 and/or within the interior of the device.

During use, the user inserts their foot into the interior of the housing and may tap their foot onto the foam base. Tapping the foam base emits the powder into the interior space of the housing such that the powder is evenly dispersed onto the foot. The tapping action emits the powder into the air such that the powder becomes airborne within the interior of the cavity to help distribute the powder into the crevices of the feet and over all surfaces of the foot (or feet).

It is to be noted that while the embodiments describe the device being used for a single foot at a time, the device may be modified to allow the user to input both feet into the interior of the housing. In such, the user can apply the foot powder to both feet at once.

In some embodiments, the device includes a zipper providing access into the interior of the housing. The zipper may enable the replacement of the foam pad as well as access to the interior of the housing for other maintenance, cleaning, refilling of the powder, etc.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A foot powder application device, comprising:
a housing defined by a top surface, a bottom, and a plurality of sidewalls;
an interior of the housing to retain a foot powder;
a lid provided on the top surface, the lid enabling housing to be opened and closed via a fastener, wherein opening the lid exposes an opening dimensioned to receive a foot; and
a foam base positioned on the bottom of the interior of the housing.

2. The foot powder application device of claim 1, wherein the foam base includes antimicrobial properties.

3. The foot powder application device of claim 1, wherein a closed configuration is defined by the fastener being engaged to close the opening.

4. The foot powder application device of claim 3, wherein the closed configuration retains the foot powder within the interior.

5. The foot powder application device of claim 1, wherein opening the lid exposes an elastic cover, and wherein the opening is provided on the elastic cover to fittingly contact an ankle region of the foot.

6. The foot powder application device of claim 5, wherein the elastic cover includes an elastic component defining an edge of the opening.

7. The foot powder application device of claim 6, wherein the elastic component is biased to fittingly engage with the ankle region.

8. The foot powder application device of claim 1, further comprising a carrying handle attached to the housing.

9. The foot powder application device of claim 1, wherein the housing is constructed to prevent the access of contaminants into the interior of the housing.

10. A foot powder application device, comprising:
a housing defined by a top surface, a bottom, and a plurality of sidewalls;
an interior of the housing to retain a foot powder;
a lid to enable the housing to be configured in an open configuration and a closed configuration, the lid provided on the top surface;
an elastic cover including an opening, the elastic cover exposable via opening the lid, the opening to enable the insertion of a foot into the interior;
a foam base positioned on the bottom of the interior of the housing,
wherein a user inserts the foot into the interior and positioned the foot on the foam base, and wherein the foot is tapped to agitate the foot powder within the interior to provide a sanitary method of applying the foot powder to the foot.

11. The foot powder application device of claim 10, wherein the foam base includes antimicrobial properties.

12. The foot powder application device of claim 10, wherein a closed configuration is defined by the fastener being engaged to close the opening.

13. The foot powder application device of claim 12, wherein the closed configuration retains the foot powder within the interior.

14. The foot powder application device of claim 13, wherein the elastic cover includes an elastic component defining an edge of the opening.

15. The foot powder application device of claim 14, wherein the elastic component is biased to fittingly engage with the ankle region.

16. The foot powder application device of claim 10, further comprising a carrying handle attached to the housing.

17. The foot powder application device of claim 10, wherein the housing is constructed to prevent the access of contaminants into the interior of the housing.

18. The foot powder application device of claim 10, wherein the housing is constructed of a waterproof material to prevent moisture from entering the interior.

19. The foot powder application device of claim 10, further comprising a storage pocket positioned on the back surface of the housing.

20. A foot powder application device, comprising:
a waterproof housing defined by a top surface, a bottom, and a plurality of sidewalls;
an interior of the housing to retain a foot powder;
a selectively openable lid to enable the housing to be configured in an open configuration and a closed configuration, the lid provided on the top surface;
a fastener to connect the lid to the top surface;
an elastic cover positioned under the lid, the elastic cover including an opening, the elastic cover exposable via opening the lid to enable the insertion of a foot into the interior;
an elastic component defining an edge of the opening, the elastic opening biased to fittingly contact an ankle region;
an antimicrobial foam base positioned on the bottom of the interior of the housing,
wherein a user inserts the foot into the interior and positioned the foot on the foam base, and wherein the foot is tapped to agitate the foot powder within the interior to provide a sanitary method of applying the foot powder to the foot.
